(19) United States
(12) Patent Application Publication
(10) Pub. No.: US 2025/0025619 A1
(43) Pub. Date: Jan. 23, 2025

(54) DRAINAGE SYSTEMS INCLUDING DISPLACEMENT BODY FOR PROVIDING A NEGATIVE PRESSURE FOR DRAINING AND COLLECTING BODILY FLUIDS

(71) Applicant: Bard Peripheral Vascular, Inc., Franklin Lakes, NJ (US)

(72) Inventors: Michael Plishka, Lake Villa, IL (US); Erin L. Schaus, Elk Grove Village, IL (US); Katherine Doniec, Deer Park, IL (US); Amy A. Mason, Vernon Hills, IL (US)

(73) Assignee: Bard Peripheral Vascular, Inc., Franklin Lakes, NJ (US)

(21) Appl. No.: 18/716,772

(22) PCT Filed: Dec. 9, 2021
(86) PCT No.: PCT/US21/62633

§ 371 Date: Jun. 5, 2024

(51) Int. Cl. A61M 1/00; A61M 39/10; A61M 39/22

(52) U.S. Cl. CPC A61M 1/84; A61M 1/98; A61M 39/10; A61M 39/22

(57) ABSTRACT

A drainage system includes a fluid-receiving container that includes an inlet and a drainage conduit communicatively coupled to the inlet of the fluid-receiving container. A first one-way valve is along the drainage conduit that provides a one-way fluid flow direction through the drainage conduit into the fluid-receiving volume. A displacement body is out-of-line with the drainage conduit and fluidly connected to the drainage conduit. The displacement body is configured to be actuated and released to provide a negative pressure to a fluid-receiving passageway through the drainage conduit. Actuation of the displacement body displaces air within the fluid-receiving passageway, the air being forced into the fluid-receiving volume by a second one-way valve and inhibited from escaping the fluid-receiving volume by the first one-way valve.

### TECHNICAL FIELD

The present specification generally relates to drainage systems and methods for draining bodily fluid and, more specifically, to drainage systems and methods including a displacement body for introducing a negative pressure for draining and collecting bodily fluids.

### BACKGROUND

The pleural cavity is the thin, fluid-filled space between the two pulmonary pleurae (known as visceral and parietal) of each lung. A pleura is a serous membrane that folds back onto itself to form a two-layered membranous pleural sac. The pleural space is normally filled with approximately five to 20 mL of serous fluid. The turnover of fluid in the pleural space is normally quite rapid—roughly 35 to 75% of the total fluid per hour, so that 5 to 10 L of fluid moves through the pleural space each day. A pleural effusion is a build-up of fluid in the pleural space. A pleural effusion may also be referred to as effusion or pulmonary effusion. The type of fluid that forms a pleural effusion may be categorized as transudate or exudate.

Peritoneal effusion or ascites refers to an excess collection of fluid in the abdominal cavity, such as more than 25 mL of fluid. The most common cause of such excess fluid collection is liver cirrhosis and other causes include cancer and pancreatitis.

There are a number of drainage systems for managing recurrent pleural effusions and ascites at home. Two such systems are the PleurX™ and PeritX™ drainage systems, commercially available from Becton, Dickinson and Company. These systems utilize a catheter and drainage bottles that collect fluid. The catheter is inserted, typically as a simple outpatient procedure, in the chest for draining pleural effusions or in the abdomen for ascites. An end of the catheter stays outside of the body, hidden under a bandage when not in use. To drain, the end of the catheter is connected to a drainage line on the bottle that is pre-evacuated to provide a negative pressure, and the negative pressure inside the bottle is used to draw out the bodily fluid.

### SUMMARY

According to a first embodiment, a drainage system for draining bodily fluid includes a fluid-receiving container having a fluid-receiving volume located therein that is configured to receive a bodily fluid. The fluid-receiving container includes an inlet and a drainage conduit communicatively coupled to the inlet of the fluid-receiving container. The drainage conduit is communicatively coupled to a connector that connects to a drainage catheter. A first one-way valve is along the drainage conduit that provides a one-way fluid flow direction through the drainage conduit into the fluid-receiving volume. A displacement body is out-of-line with the drainage conduit and fluidly connected to the drainage conduit. The displacement body is configured to be actuated and released to provide a negative pressure to a fluid-receiving passageway through the drainage conduit. The first one-way valve inhibits air from being drawn from the fluid-receiving volume of the fluid-receiving container due to the negative pressure. A second one-way valve is along the fluid-receiving passageway at a location between the displacement body and the connector that inhibits fluids from passing in a direction toward the connector. Actuation of the displacement body displaces air within the fluid-receiving passageway, the air being forced into the fluid-receiving volume by the second one-way valve and inhibited from escaping the fluid-receiving volume by the first one-way valve.

According to a second embodiment, a method of forming a drainage system for draining a bodily fluid from a body cavity is provided. The method includes fluidly connecting an outlet end of an drainage conduit to an inlet of a fluid-receiving container and fluidly connecting a first one-way valve to the drainage conduit. The one-way valve provides a one-way fluid flow direction through the drainage conduit into a fluid-receiving volume of the fluid-receiving container. A displacement body is fluidly connected to the drainage conduit in an out-of-line fashion. A connector is fluidly connected to an inlet end of the drainage conduit, the connector configured to connect to a drainage catheter. The displacement body is configured to be actuated manually to provide a negative pressure to a fluid-receiving passageway through the drainage conduit, the one-way valve inhibiting air from being drawn from the fluid-receiving volume of the fluid-receiving container due to the negative pressure.

According to a third embodiment, a drainage system for draining bodily fluid includes a fluid-receiving container having a fluid-receiving volume located therein that is configured to receive a bodily fluid, the fluid-receiving container comprising an inlet. A drainage conduit is communicatively coupled to the inlet of the fluid-receiving container. The drainage conduit is communicatively coupled to a connector that connects to a drainage catheter. A first one-way valve provides a one-way fluid flow direction through the drainage line and into the fluid-receiving volume. A displacement body is out-of-line with the drainage conduit and fluidly connected to the drainage conduit. The displacement body is configured to be actuated and released to provide a negative pressure to a fluid-receiving passageway through the drainage conduit. The displacement body is located between the first one-way valve and a second one-way valve. Compressing the displacement body displaces air within the displacement body. The second one-way valve prevents air from entering the displacement body. The first one-way valve allows a negative pressure to be communicated from the displacement body, through the drainage conduit and to the connector.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a drainage system including a displacement body, according to one or more embodiments shown and described herein;

FIG. 2A schematically depicts the drainage system of FIG. 1 in operation, according to one or more embodiments shown and described herein;

FIG. 2B schematically depicts the drainage system of FIG. 1 in operation, according to one or more embodiments shown and described herein;

FIG. 2C schematically depicts the drainage system of FIG. 1 in operation, according to one or more embodiments shown and described herein;

FIG. 3 schematically depicts a portion of another drainage system including displacement body, according to one or more embodiments shown and described herein;

FIG. 4 schematically illustrates a portion of another drainage system including displacement member, according to one or more embodiments shown and described herein;

FIG. 5 schematically depicts a portion of another drainage system including displacement member, according to one or more embodiments shown and described herein; and

FIG. 6 schematically depicts the drainage system of FIG. 5 in operation, according to one or more embodiments shown and described herein.

### DETAILED DESCRIPTION

Embodiments described herein are generally directed to drainage systems for draining bodily fluids. The drainage systems include a fluid-receiving container having a fluid-receiving volume that receives a bodily fluid therein through an inlet. The fluid-receiving container is in fluid communication with a drainage conduit connected to the fluid-receiving container at the inlet. The drainage conduit is fluidly connected to a connector that connects to a drainage catheter. A displacement body is fluidly connected to the drainage conduit by a connecting conduit such that the displacement body is out-of-line with the drainage conduit. The displacement body is configured to be actuated and released to provide a negative pressure to a fluid-receiving passageway through the drainage conduit. A one-way valve is located along the drainage conduit downstream of the connecting conduit. The one-way valve inhibits air from being drawn from the fluid-receiving volume of the fluid-receiving container due to the negative pressure. Another one-way valve is located along the drainage conduit at a location between the connecting conduit and the connector that inhibits fluids from passing in a direction toward the connector. Actuation of the displacement body displaces air within the fluid-receiving passageway, the air being forced into the fluid-receiving volume by the another one-way valve and inhibited from escaping the fluid-receiving volume by the one-way valve downstream of the connecting catheter.

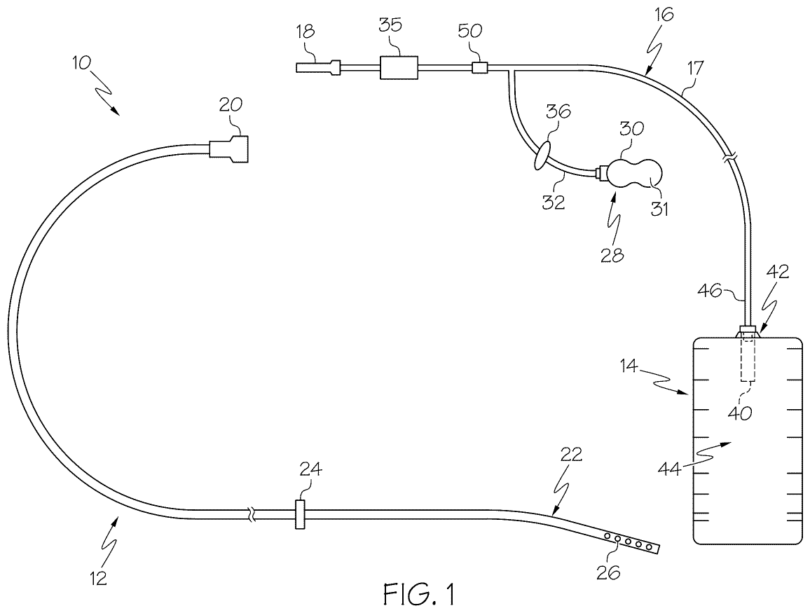

Referring to FIG. 1, a drainage system 10 for use in draining bodily fluids from body cavities includes a drainage catheter 12 that may be partially implanted in a patient's body and a fluid-receiving container 14 that can be fluidly connected to the drainage catheter 12. The fluid-receiving container 14 may be connected to the drainage catheter 12 by a drainage conduit 16 that extends from the fluid-receiving container 14 to the drainage catheter 12 providing a fluid-receiving passageway 17 therebetween. In some embodiments, the drainage conduit 16 may include a connector 18 that connects to a valve 20. As an example, the valve 20 may be a two-piece valve that is normally closed, but that opens with insertion of the connector 18 to allow bodily fluid to flow through the drainage conduit 16. A distal end portion 22 of the drainage catheter 12 may be implanted in the patient's body for pleural or peritoneal drainage or other body location for draining a bodily fluid. In some embodiments, the distal end portion 22 may include a sealing cuff 24 and fenestrations 26 through which the bodily fluid may enter the drainage catheter 12 and be carried from the pleural, peritoneal or other cavity space.

A displacement body 28 includes a squeeze bulb 31 that is used to supply negative pressure through the drainage conduit 16 and the drainage catheter 12. The negative pressure provides a suction to automatically draw bodily fluid from the cavity in which the drainage catheter 12 is inserted toward the fluid-receiving container 14. As used herein, the terms “negative pressure,” “suction,” vacuum” and “partial vacuum” may be used interchangeably and refer to a force over an area produced by a difference in pressure. Pressure may be referred to as “negative” in reference to atmospheric pressure (i.e., gauge pressure). It should be noted that while using the displacement body for draining bodily fluids from body cavities is described primarily herein, the displacement bodies may be used to drain bodily fluids from other locations, such as wounds.

The displacement body 28 includes a resiliently flexible outer wall 30 that forms a bulb-shape having an internal volume that is filled with a gas, such as air. The flexible outer wall 30 can be formed of any suitable compliant material, such as silicone. The flexible outer wall 30 can be constricted (e.g., squeezed) and, when released, used to generate the negative pressure differential relative to pressure within the body cavity to initiate fluid drainage from the body cavity. The displacement body 28 is fluidly connected to a connecting conduit 32 that is fluidly connected to the drainage conduit 16. In some embodiments, the drainage conduit 16 and the connecting conduit 32 may be part of a same T or Y-shaped tubing. In other embodiments, a connection, such as a T or Y-shaped connector may be used to fluidly connect the drainage conduit 16 and the connecting conduit 32. A flow blocking member 36, such as a filter (e.g., hydrophobic or hydrophilic) or one-way valve may be located in the connecting conduit 32 between the displacement body 28 and the drainage conduit 16. The flow blocking member 36 can allow the gas (air) to pass therethrough and through the connecting conduit 32, at least until being wetted by bodily fluid, and block drainage fluid from passing therethrough and entering the displacement body 28 during a drainage operation. In this regard, the displacement body 28 may be considered “out-of-line” with or branched-off from the drainage conduit 16 as bodily fluid does not pass through the displacement body 28 on its way to the fluid-receiving container 14. The displacement body 28 is not fluidly connected directly to the drainage conduit 16 and fluid is blocked from the displacement body 28 and does not pass through the displacement body 28.

A one-way valve 40 is located along the drainage conduit 16 downstream of the displacement body 28. The terms “downstream” and “upstream” are with reference to drainage flow during a drainage operation with a downstream direction being toward the fluid-receiving container 14 and upstream direction being away from the fluid-receiving container. In the illustrated example, the one-way valve 40 is a flutter valve that is located at an inlet 42 to a fluid-receiving volume 44 of the fluid-receiving container 14. An end 46 of the drainage conduit 16 may be fluidly connected to the one way valve 40 at the inlet 42. In some embodiments, the end 46 may be connected to a stub tube. While the one-way valve 40 is located at the inlet 42, a one way valve may be located anywhere along the drainage conduit 16 between the fluid-receiving volume 44 and the connecting conduit 32. For example, it may be desirable to provide the one way valve 40 closer to the connecting conduit 32 to reduce a distance between the connector 18 and the one-way valve 40 thereby reducing an air-filled volume in the drainage conduit 16 when providing negative pressure, as will be described in greater detail below.

Another one-way valve 50 is located along the drainage conduit 16 upstream of the connecting conduit 32. The one-way valve 50 may be located anywhere along the drainage conduit 16 between the displacement body 28 and the connector 18. During a drainage operation, the one-way valve 50 prevents the flow of fluids (i.e., gases and liquids) upstream toward the body cavity. Further, a flow control device 35 may be used to constrict or release the drainage conduit 16, which can be used to decrease or increase fluid flow rate as desired by the user.

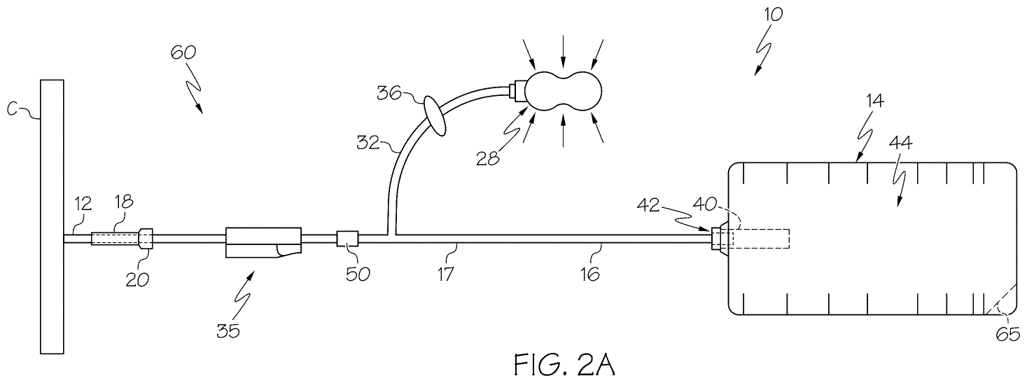

Referring also to FIGS. 2A-2C, operation of the drainage system 10 is illustrated. Once the connector 18 is fluidly connected to the drainage catheter 12 through the valve 20, a drainage operation may be initiated with the drainage catheter 12 already implanted within a body cavity represented by element C. To initiate the drainage operation, the fluid-receiving container 14 (e.g., a bag) may be placed on a floor such that it is lower than the body cavity C. Depending on locations and orientations of the body cavity C, the drainage conduit 16, the fluid-receiving container 14 and a pressure difference between the body cavity C and outside atmosphere, drainage flow may begin without actuation of the displacement body 28. This is considered gravity flow.

In some instances, it may be desirable to introduce a lower pressure within the fluid-receiving passageway 17 of the drainage catheter 12 to initiate or increase bodily fluid flow. To this end, the displacement body 28 may be manually squeezed thereby reducing its internal volume and forcing air out of the displacement body 28, through the flow blocking member 36 and into the fluid-receiving passageway 17 at step 60 (FIG. 2A). Due to the presence of the one-way valve 50 upstream of the connecting conduit 32 preventing the flow of air thereby in the upstream direction, the air displaced within the fluid-receiving passageway 17 is forced through the one-way valve 40 at the inlet 42 and into the fluid-receiving volume 44 in a process referred to as venting. The fluid-receiving container 14 may be inflated slightly and the one-way valve 40 prevents the flow of the trapped air out of the fluid-receiving volume 44.

At step 62, the displacement body 28 is manually released (FIG. 2B). Because the displacement body 28 is resilient, the displacement body 28 attempts to expand thereby creating a suction that is communicated through the connecting conduit 32 and to the fluid-receiving passageway 17. Again, presence of the one-way valve 40 prevents the air from being drawn from the fluid-receiving volume 44 and back into the displacement body 28. For this reason and because the flow blocking member 36 prevents liquid from passing therethrough and also gasses once wetted, the displacement body 28, in this embodiment, may not fully re-inflate. The other one-way valve 50 allows the negative pressure to be communicated therethrough to the drainage catheter 12.

At step 64, some of the bodily fluid may travel toward the displacement body 28 along the connecting conduit 32 (FIG. 2C). The flow blocking member 36 prevents the flow of liquid into the displacement body 28. At this point, the drainage of the bodily fluid may occur under the influence of gravity without negative pressure being introduced to the fluid-receiving passageway 17 by the displacement body 28. The flow control device 35 may be used to constrict or release the drainage conduit 16, which can be used to decrease or increase fluid flow rate as desired by the user. The fluid-receiving container 14 may be formed of a plastic sheet material that includes a line of weakness 65 that facilitates tearing of the fluid-receiving container 14 and to pour out the contents.

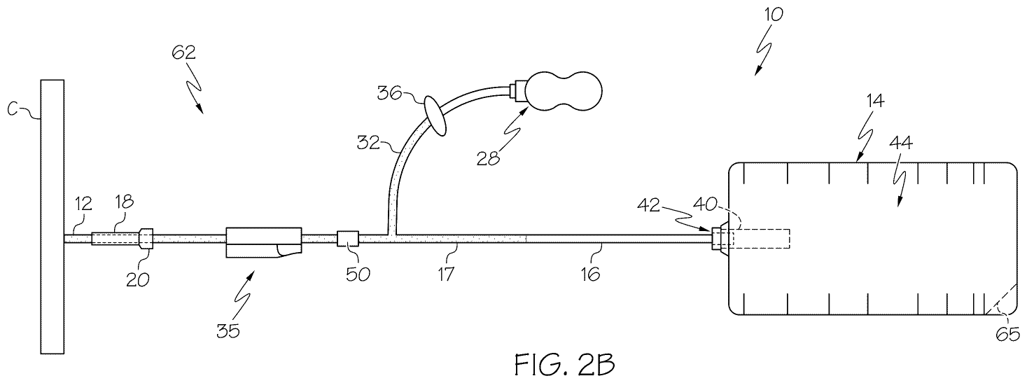

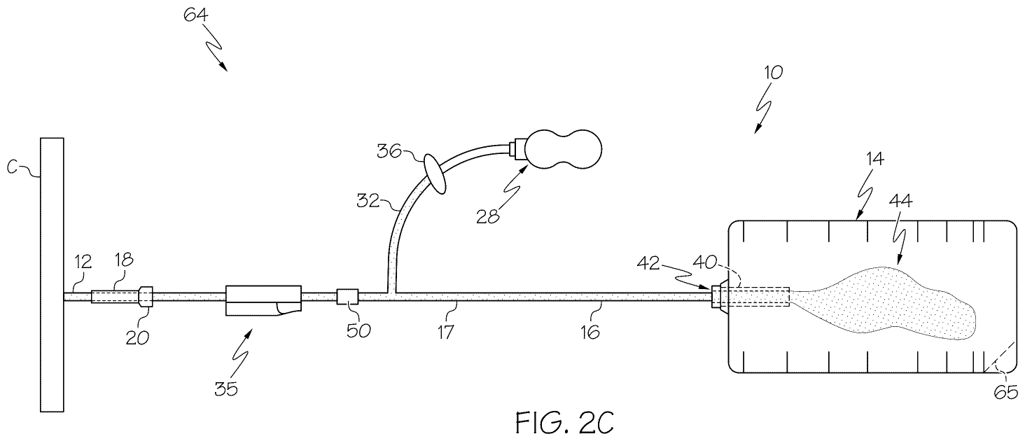

While FIGS. 1-2C show the valves 40 and 50 along the drainage conduit 16, other valve configurations are possible. For example, referring to FIG. 3, another drainage system 70 includes a displacement body 72 that is fluidly connected at an inlet 74 to a connecting conduit 76 fluidly connected to a drainage conduit 78. In this example, the drainage conduit 78 and the connecting conduit 76 are fluidly connected by a T-shaped connector 80. A flow blocking member 82, such as a filter (e.g., hydrophobic or hydrophilic) is located in the connecting conduit 76 between the displacement body 72 and the drainage conduit 78. The flow blocking member 82 can allow the air to pass therethrough and through the connecting conduit 76. The flow blocking member 82 also blocks drainage fluid from entering the displacement body 72 during a drainage operation (FIG. 4).

A one-way valve 84 is located along the connecting conduit 76 at the inlet 74 between the displacement body 72 and the flow blocking member 82. Another one-way valve 86 is located at an outlet 88 of the displacement body 72, between the displacement body 72 and the environment. In operation, manually constricting the displacement body 72 reduces the volume inside the displacement body 72. As the volume decreases, the one-way valve 84 prevents the air, represented by arrow 90, from passing therethrough and through the connecting conduit 76. The one-way valve 86 allows air, represented by arrow 92, to escape from the volume of the displacement body 72 and into the surroundings.

Referring now to FIG. 4, when the displacement body 72 is released, air from the surroundings, represented by arrow 94, is prevented by the one-way valve 86 from entering the displacement body 72. Because the displacement body 72 is resilient, the displacement body 72 attempts to expand thereby creating a suction that is communicated through the connecting conduit 76 and to fluid-receiving passageway 100. Presence of the one-way valve 86 prevents the air from being drawn from the surroundings and back into the displacement body 72. For this reason, the displacement body 72, in this embodiment, may not fully re-inflate. The other one-way valve 84 allows the negative pressure to be communicated therethrough to the fluid-receiving passageway 100 until drainage begins, as represented by arrow 102. The flow blocking member 82 prevents liquid from passing therethrough and also gasses once wetted as represented by the hatching.

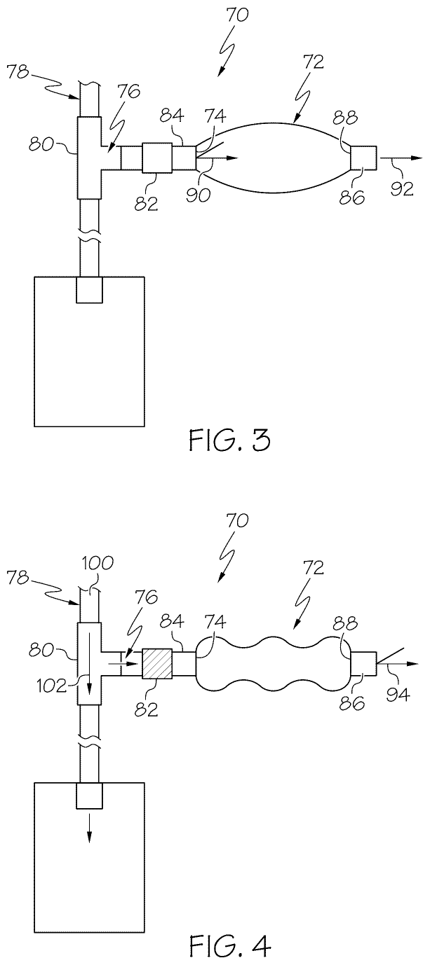

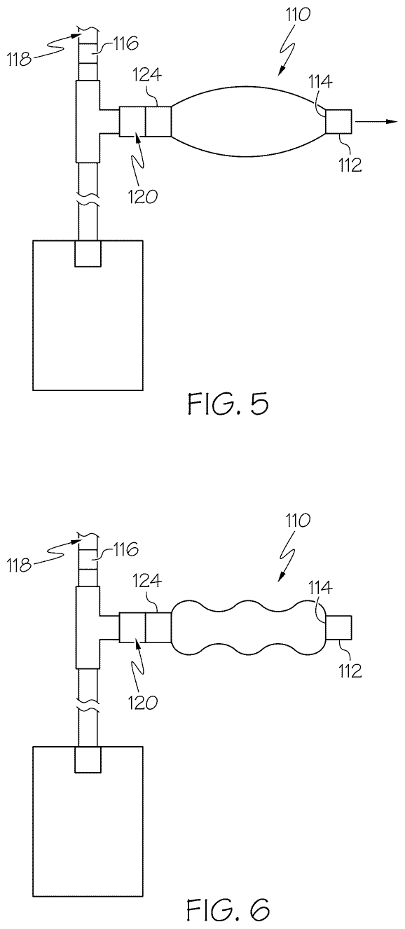

FIGS. 5 and 6 illustrate another embodiment of a displacement body 110 that includes a one-way valve 112 at an outlet location 114. As above with FIG. 4, the one-way valve 112 allows air to escape the displacement body 110 when the displacement body 110 is compressed and prevents air from entering when the displacement body 110 is released. Another one-way valve 116 is located along drainage conduit 118 upstream of the connecting conduit 120. The one-way valve 112 may be located anywhere along the drainage conduit 118 between the displacement body 110 and connector (FIG. 1). As above in FIG. 2, the one-way valve 116 prevents the flow of air and liquid upstream toward the body cavity. Releasing the displacement body 110 introduces negative pressure into the drainage conduit 118 as discussed above. A flow blocking member 124 prevents drainage liquids from entering the displacement body 110.

The above-described drainage systems may be suitable for pleural and peritoncal drainage operations and include a displacement body that can be used to enhance flow of bodily fluid from a body cavity and into a fluid-receiving container, such as a bag formed of a clear plastic film that has printed volume markings. Once the drainage conduit is attached to the drainage catheter, fluid may begin to flow without assistance from the displacement body, which is considered gravity drainage. When the displacement body is compressed manually, the displacement body pushes air out of its volume. The air may be pushed into the fluid-receiving container or into the surroundings depending on the configuration of the system. In either case, the displacement body provides a suction to the fluid-receiving passageway that passes through the drainage conduit. A flow control device may be provided that allows for manual control of the drainage rate, which can improve patient comfort during the drainage operation.

Embodiments can be described with reference to the following numbered clauses, with certain features laid out in the dependent clauses:
Clause 1: A drainage system for draining bodily fluid, the drainage system comprising: a fluid-receiving container having a fluid-receiving volume located therein that is configured to receive a bodily fluid, the fluid-receiving container comprising an inlet; a drainage conduit communicatively coupled to the inlet of the fluid-receiving container and having a fluid passageway, the drainage conduit communicatively coupled to a connector for connecting to a drainage catheter; a first one-way valve along the drainage conduit for inhibiting fluid flow through the drainage conduit out of the fluid-receiving volume; a displacement body that is at a branched-off position relative to the drainage conduit and is fluidly connected to the fluid passageway of the drainage conduit, the displacement body configured to be actuated and released to provide a negative pressure to the fluid passageway through the drainage conduit; and a second one-way valve in the fluid passageway of the drainage conduit downstream of the connector, the second one-way valve positioned for inhibiting fluids from passing in a direction toward the connector; wherein actuation of the displacement body displaces air within the fluid passageway, the air being forced into the fluid-receiving volume by the second one-way valve and inhibited from escaping the fluid-receiving volume by the first one-way valve.
Clause 2: The drainage system of clause 1 further comprising a hydrophobic or hydrophilic filter located between the displacement body and the drainage conduit that inhibits liquid from entering the displacement body.
Clause 3: The drainage system of clause 2 further comprising a flow control device located along the drainage conduit that adjusts a diameter of the fluid passageway.
Clause 4: The drainage system of any one of clauses 1-3, wherein the displacement body is fluidly connected to the drainage conduit by a connecting conduit located between the first one-way valve and the second one-way valve.
Clause 5: The drainage system of any one of clauses 1-3, wherein the displacement body comprises a squeeze bulb that is open at an outlet location where air exits the displacement body when compressed and an inlet location where air enters the displacement body from the fluid-receiving passageway when released.
Clause 6: The drainage system of any one of clauses 1-5, wherein the fluid-receiving container is a bag comprising a line of weakness that facilitates tearing the bag.
Clause 7: The drainage system of any one of clauses 1-4, wherein the displacement body comprises a squeeze bulb that is only open to the drainage conduit.
Clause 8: A method of forming a drainage system for draining a bodily fluid from a body cavity, the method comprising: fluidly connecting an outlet end of an drainage conduit to an inlet of a fluid-receiving container and fluidly connecting a first one-way valve to the drainage conduit, the one-way valve providing a one-way fluid flow direction through the drainage conduit into a fluid-receiving volume of the fluid-receiving container; fluidly connecting a displacement body to the drainage conduit in an out-of-line fashion; and fluidly connecting a connector to an inlet end of the drainage conduit, the connector configured to connect to a drainage catheter;
wherein the displacement body is configured to be actuated manually to provide a negative pressure to a fluid-receiving passageway through the drainage conduit, the one-way valve inhibiting air from being drawn from the fluid-receiving volume of the fluid-receiving container due to the negative pressure.
Clause 9: The method of clause 8 further comprising placing another one-way valve along the fluid-receiving passageway at a location between the displacement body and the connector that inhibits fluid from passing in a direction toward the connector.
Clause 10: The method of clause 9 further comprising placing a hydrophobic or hydrophilic filter between the displacement body and the drainage conduit that inhibits liquid from entering the displacement body.
Clause 11: The method of clause 10 further comprising placing a flow control device along the drainage conduit that adjusts a diameter of the fluid-receiving passageway.
Clause 12: The method of clause 11, wherein the flow control device is located between the another one-way valve and the connector.
Clause 13: The method of any one of clauses 9-12, wherein the displacement body is fluidly connected to the drainage conduit by a connecting conduit located between the one-way valve and the another one-way valve.
Clause 14: The method of any one of clauses 8-13, wherein the fluid-receiving container is a bag comprising a line of weakness that facilitates tearing the bag.
Clause 15: The method of any one of clauses 8-14, wherein the displacement body comprises a squeeze bulb that is only open to the drainage conduit.
Clause 16: A drainage system for draining bodily fluid, the drainage system comprising: a fluid-receiving container having a fluid-receiving volume located therein that is configured to receive a bodily fluid, the fluid-receiving container comprising an inlet; a drainage conduit communicatively coupled to the inlet of the fluid-receiving container, the drainage conduit being communicatively coupled to a connector that connects to a drainage catheter; a first one-way valve that provides a one-way fluid flow direction through the drainage line and into the fluid-receiving volume; and a displacement body that is out-of-line with the drainage conduit and fluidly connected to the drainage conduit, the displacement body configured to be actuated and released to provide a negative pressure to a fluid-receiving passageway through the drainage conduit, wherein the displacement body is located between the first one-way valve and a second one-way valve; wherein compressing the displacement body displaces air within the displacement body, the second one-way valve preventing air from entering the displacement body, the first one way valve allowing a negative pressure to be communicated from the displacement body, through the drainage conduit and to the connector.
Clause 17: The drainage system of clause 16, wherein the air being forced into the fluid-receiving volume by the second one-way valve and inhibited from escaping the fluid-receiving volume by the first one-way valve.
Clause 18: The drainage system of clause 16 or 17 further comprising a hydrophobic or hydrophilic filter located between the displacement body and the drainage conduit that inhibits liquid from entering the displacement body.
Clause 19: The drainage system of clause 17 or 18 further comprising a flow control device located along the drainage conduit that adjusts a diameter of the fluid-receiving passageway.
Clause 20: The drainage system of any of clauses 16-19, wherein the displacement body comprises a squeeze bulb that is open at an outlet location where air exits the displacement body when compressed and an inlet location where air enters the displacement body from the fluid-receiving passageway when released.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A drainage system for draining bodily fluid, the drainage system comprising:
a fluid-receiving container having a fluid-receiving volume located therein that is configured to receive a bodily fluid, the fluid-receiving container comprising an inlet;
a drainage conduit communicatively coupled to the inlet of the fluid-receiving container and having a fluid passageway, the drainage conduit communicatively coupled to a connector for connecting to a drainage catheter;
a first one-way valve along the drainage conduit for inhibiting fluid flow through the drainage conduit out of the fluid-receiving volume;
a displacement body that is at a branched-off position relative to the drainage conduit and is fluidly connected to the fluid passageway of the drainage conduit, the displacement body configured to be actuated and released to provide a negative pressure to the fluid passageway through the drainage conduit; and
a second one-way valve in the fluid passageway of the drainage conduit downstream of the connector, the second one-way valve positioned for inhibiting fluids from passing in a direction toward the connector;
wherein actuation of the displacement body displaces air within the fluid passageway, the air being forced into the fluid-receiving volume by the second one-way valve and inhibited from escaping the fluid-receiving volume by the first one-way valve.

2. The drainage system of claim 1 further comprising a hydrophobic or hydrophilic filter located between the displacement body and the drainage conduit that inhibits liquid from entering the displacement body.

3. The drainage system of claim 2 further comprising a flow control device located along the drainage conduit that adjusts a diameter of the fluid passageway.

4. The drainage system of claim 1, wherein the displacement body is fluidly connected to the drainage conduit by a connecting conduit located between the first one-way valve and the second one-way valve.

5. The drainage system of claim 1, wherein the displacement body comprises a squeeze bulb that is open at an outlet location where air exits the displacement body when compressed and an inlet location where air enters the displacement body from the fluid passageway when released.

6. The drainage system of claim 1, wherein the fluid-receiving container is a bag comprising a line of weakness that facilitates tearing the bag.

7. The drainage system of claim 1, wherein the displacement body comprises a squeeze bulb that is only open to the drainage conduit.

8. A method of forming a drainage system for draining a bodily fluid from a body cavity, the method comprising:
fluidly connecting an outlet end of an drainage conduit to an inlet of a fluid-receiving container and fluidly connecting a first one-way valve to the drainage conduit, the one-way valve providing a one-way fluid flow direction through the drainage conduit into a fluid-receiving volume of the fluid-receiving container;
fluidly connecting a displacement body to the drainage conduit in an out-of-line fashion; and
fluidly connecting a connector to an inlet end of the drainage conduit, the connector configured to connect to a drainage catheter;
wherein the displacement body is configured to be actuated manually to provide a negative pressure to a fluid-receiving passageway through the drainage conduit, the one-way valve inhibiting air from being drawn from the fluid-receiving volume of the fluid-receiving container due to the negative pressure.

9. The method of claim 8 further comprising placing another one-way valve along the fluid-receiving passageway at a location between the displacement body and the connector that inhibits fluid from passing in a direction toward the connector.

10. The method of claim 9 further comprising placing a hydrophobic or hydrophilic filter between the displacement body and the drainage conduit that inhibits liquid from entering the displacement body.

11. The method of claim 10 further comprising placing a flow control device along the drainage conduit that adjusts a diameter of the fluid-receiving passageway.

12. The method of claim 11, wherein the flow control device is located between the another one-way valve and the connector.

13. The method of claim 9, wherein the displacement body is fluidly connected to the drainage conduit by a connecting conduit located between the one-way valve and the another one-way valve.

14. The method of claim 8, wherein the fluid-receiving container is a bag comprising a line of weakness that facilitates tearing the bag.

15. The method of claim 8, wherein the displacement body comprises a squeeze bulb that is only open to the drainage conduit.

16. A drainage system for draining bodily fluid, the drainage system comprising:
a fluid-receiving container having a fluid-receiving volume located therein that is configured to receive a bodily fluid, the fluid-receiving container comprising an inlet;
a drainage conduit communicatively coupled to the inlet of the fluid-receiving container, the drainage conduit being communicatively coupled to a connector that connects to a drainage catheter;
a first one-way valve that provides a one-way fluid flow direction through the drainage line and into the fluid-receiving volume; and
a displacement body that is out-of-line with the drainage conduit and fluidly connected to the drainage conduit, the displacement body configured to be actuated and released to provide a negative pressure to a fluid-receiving passageway through the drainage conduit;
wherein the displacement body is located between the first one-way valve and a second one-way valve;
wherein compressing the displacement body displaces air within the displacement body, the second one-way valve preventing air from entering the displacement body, the first one-way valve allowing a negative pressure to be communicated from the displacement body, through the drainage conduit and to the connector.

17. The drainage system of claim 16, wherein the air being forced into the fluid-receiving volume by the second one-way valve and inhibited from escaping the fluid-receiving volume by the first one-way valve.

18. The drainage system of claim 16 further comprising a hydrophobic or hydrophilic filter located between the displacement body and the drainage conduit that inhibits liquid from entering the displacement body.

19. The drainage system of claim 17 further comprising a flow control device located along the drainage conduit that adjusts a diameter of the fluid-receiving passageway.

20. The drainage system of claim 16, wherein the displacement body comprises a squeeze bulb that is open at an outlet location where air exits the displacement body when compressed and an inlet location where air enters the displacement body from the fluid-receiving passageway when released.
